(19) United States
(12) Patent Application Publication
(10) Pub. No.: US 2007/0074762 A1
(43) Pub. Date: Apr. 5, 2007

(54) Internal seal for medical instrument

(75) Inventor: Pavel Menn, Marblehead, MA (US)

Correspondence Address: GREENBLUM & BERNSTEIN, P.L.C., 1950 ROLAND CLARKE PLACE, RESTON, VA, 20191, US

(73) Assignee: MICROLINE PENTAX Inc., Beverly, MA (US)

(21) Appl. No.: 11/195,718

(22) Filed: Aug. 3, 2005

(51) Int. Cl. F16K 13/00; F16K 13/10; F16L 43/00; F16L 55/24; A61M 5/178; A61M 5/14; A61M 5/00

(52) U.S. Cl. 137/247.13; 604/167.01; 604/256

(57) ABSTRACT

An elastomeric internal seal for preventing or reducing fluid from passing from a first end of a tubular instrument to a second end of the tubular instrument. The seal has an edge portion configured to contact an inner surface of the instrument, at least one receiving aperture configured to receive an apparatus therethrough, the apparatus extending in a generally longitudinal direction in relation to the tubular instrument. The seal further has a lip portion channel generally extending toward the first end and about a circumference of the at least one receiving aperture, wherein the first lip portion is configured such that pressure created by the fluid causes the first lip portion to press against the apparatus.

### BACKGROUND OF THE INVENTION

1. Field of the Invention

The present invention is related to a seal, and more particularly, to an internal seal used in a medical device for stapling a patient's body to prevent or minimize fluid in a patient's body from getting into the mechanism of the medical device.

2. Background and Material Information

In medical devices used in a patient's body, it is desirable to prevent air, liquid, or other fluid in the body from entering far enough into the device to reach and possibly harm the mechanisms of the device.

Prevention or reduction of gas or fluid leakage into medical devices or mechanisms is often accomplished by the inclusion of a seal in the medical device. This seal is placed between the point at which the device touches the body and thus is open to fluid or air entry and the point at which the mechanisms of the device are located. The related art generally involves a rubber disc with an opening for parts of the device to move through. On either side of the rubber disc, a metal plate is attached. This type of seal creates multiple problems. As pressure from fluid varies across the seal, the rubber tends to bulge out beyond the metal plates at certain points and not at others thereby creating an uneven or vulnerable seal. The metal plates require attachment to the rubber disc using a fastening mechanism thus problems can arise if the fastening mechanism breaks or problems can arise due to the extra holes in the rubber seal needed for the fastening mechanism which present more possible areas of fluid or air leakage.

### SUMMARY OF THE INVENTION

The present application provides an internal seal for preventing fluid from passing from a first end of a tubular instrument to a second end of the tubular instrument. The seal may have an edge portion configured to contact an inner surface of the instrument with at least one receiving aperture to receive an apparatus therethrough, the apparatus extending in a generally longitudinal axial direction in relation to the tubular instrument, and a generally V-shaped channel generally extending about a circumference of the receiving aperture. The channel may be configured such that pressure created by the fluid causes one wall of the channel to press against the apparatus thus creating a seal.

Both walls of each V-shaped channel may be obliquely angled in relation to the generally longitudinal direction. Additionally, the receiving aperture may be configured to allow the apparatus to move in the axial direction. Further, the seal may include an ethylene propylene diene monomer (EPDM).

Another feature may be a generally V-shaped edge channel generally extending about the edge portion of the seal, so that pressure created by the fluid causes one wall of the edge channel to press against the inner surface of the instrument.

The invention further may feature a tapered lip generally extending about the edge portion of the seal so that pressure created by the fluid causes the tapered lip to press against the inner surface of the instrument.

The internal seal may also comprise a first lip portion channel generally extending toward the first end and about a circumference the receiving apertures, where the first tapered lip portion is configured such that pressure created by the fluid causes the first lip portion to press against the apparatus, the first lip portion also may be tapered or obliquely angled in relation to the generally longitudinal direction. Also, the receiving aperture may allow the apparatus to move in the axial direction.

Additionally, a second lip portion may generally extend toward the first end and about a circumference of and facing the first lip portion. Further, at least one of either the first lip portion or second lip portion may be tapered or obliquely angled in relation to the generally longitudinal direction and the two lip portions form a generally V-shaped channel.

The invention may also feature an edge lip, generally extending about the edge portion, where pressure created by the fluid causes the edge lip to press against the inner surface of the instrument, with the edge lip being tapered.

The present application also provides a medical instrument with a tubular portion having a first end, which may be inserted into a body cavity, and a second end, where an apparatus may be generally disposed within the tubular portion and extend in a generally longitudinal direction in relation to the tube in order to effect an operation relating to the body cavity. The medical device further may include an internal seal in the tubular portion, which prevents fluid from passing from the first end to second end of the device. The seal may include an edge portion that contacts an inner surface of the tubular portion, at least one receiving aperture in order to receive the apparatus therethrough, and a first lip portion channel generally extending toward the first end about the circumference of the receiving apertures such that pressure created by the fluid from the body cavity causes the first tapered lip portion to press against the apparatus.

The seal may also feature a second lip portion generally extending toward the first end about the circumference of and facing the first lip portion. Further, at least one of the first lip portion or the second lip portion may be tapered.

Other exemplary embodiments and advantages of the present invention may be ascertained by reviewing the present disclosure and the accompanying drawings, and the above description should not be considered to limit the scope of the present application.

### BRIEF DESCRIPTION OF THE DRAWINGS

The present invention is further described in the detailed description which follows, in reference to the noted plurality of drawings by way of non-limiting examples of preferred embodiments of the present invention, in which like numerals represent like elements throughout the several views of the drawings, wherein:

FIG. 1 is a perspective view of an internal seal for an instrument according to an embodiment of the present invention;

FIG. 2 is a side cross-sectional view the seal of FIG. 1 inside the instrument;

FIG. 3 is an enlarged side cross-sectional view of the seal inside the instrument;

FIG. 4 is a perspective view of a seal according to a second embodiment of the present invention;

FIG. 5 is a side cross-sectional view of the seal of FIG. 4;

FIG. 6 is a perspective view of a seal according to a third embodiment of the present invention;

FIG. 7 is a side cross-sectional view of the seal of FIG. 6;

FIG. 8 is a perspective view of a seal according to a fourth embodiment of the present invention; and

FIG. 9 is a side cross-sectional view of the seal of FIG. 8.

### DETAILED DESCRIPTION OF THE PREFERRED EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

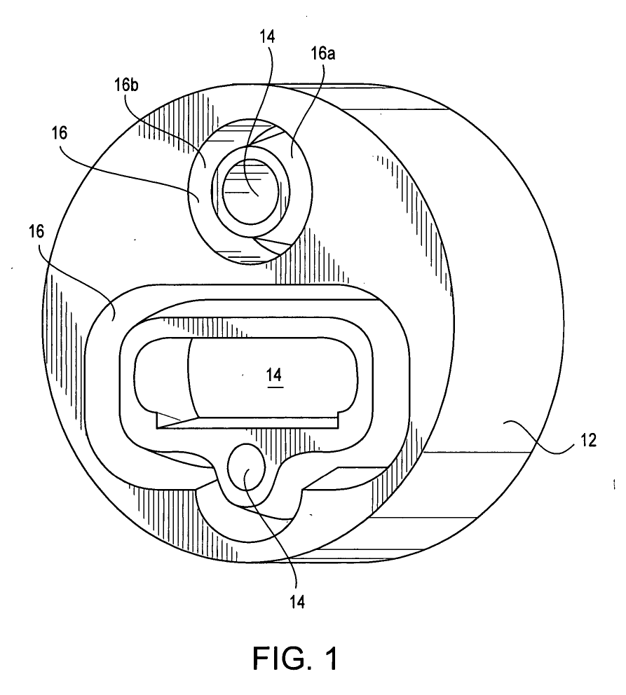

Referring to the drawings wherein like characters represent like elements, FIGS. 1 to 7 show an internal seal 10, 110, 210 for preventing or minimizing fluid from passing from a first end of a device 22 to a second end of the device according to an embodiment of the present invention. The seal 10, 110, 210 is particularly useful in medical devices that are inserted into a body cavity for preventing fluid (e.g., gas, blood or other fluids) from the body cavity from traveling into the mechanical parts of the medical device 22, but may also be used in a variety of other devices or instruments where it is necessary to prevent fluid or air from traveling beyond a certain location. The seal 10, as shown in FIG. 1, may include a circumferential edge surface 12 which is in contact with and extends about a circumference of the inner surface of the device 22, the edge surface extending in a direction generally parallel to a longitudinal axial direction X of the device.

As shown in FIG. 1, the seal includes at least one receiving aperture 14 through which an apparatus (including but not limited to a clip applier cartridge C and/or an operating rod R) can move in the longitudinal axial direction X. The seal further includes a channel 16 around each receiving aperture 14. The seal shown in the figures is round, however, it should be understood by those skilled in the art that the seal can be made any shape to fit the device 22. The device 22 shown in the figures is round and tubular, although it should be understood by those skilled in the art that the device can have a wide variety of configurations, e.g., such as square tubular and spherical. The channels 16 are defined by an inner (first) lip portion 16a extending about the circumference of the receiving aperture 14, and by an outer (second) lip portion 16b facing the inner lip portion 16b. In other words, the inner lip portion 16a and outer lip portion 16b form the walls of the channels 16. To enhance the sealing of the aperture 14, at least one lip portion 16a, 16b may be angled or tapered. For example, the inner lip 16a portion may form a frusto-conical shape around the aperture, such that fluid flowing toward the channel 16 causes the fluid to press against the inner lip portion 16a generally in radial direction P, such that the inner lip portion 16a further presses against the apparatus C, R to increase the sealing properties of the seal 10. Similarly, the outer lip portion 16b may form a frusto-conical shape at generally the same angle as the inner lip portion 16a, but in an opposite direction to substantially form a mirror image angle with the inner lip portion (thereby substantially forming a V-shape with inner lip portion 16a). Thus, fluid flowing toward the channel 16 causes the outer lip portion 16b to press in an outward direction toward the inner surface of the device 22. While the figures show an uninterrupted channel 16 extending around the apertures 14, it should be understood by those skilled in the art that in any of the embodiments described herein, that the channel may be interrupted or discontinuous.

Alternatively, the outer and inner lip portions 16a, 16b may form different angles with respect to each other and/or only one lip portion 16a, 16b may be angled (while the other is substantially parallel to the axial direction X). Also, neither lip portion 16a, 16b may be angled. The channels are shown in a generally V shaped configuration, but as described herein, it should be understood by those skilled in the art that “V shaped” broadly includes channels extending in a V, U, J, three-sided square, any combination thereof, and the like.

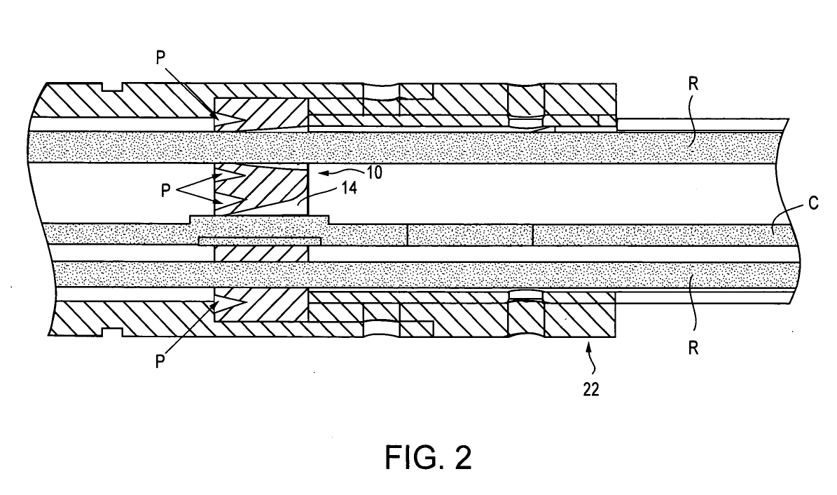

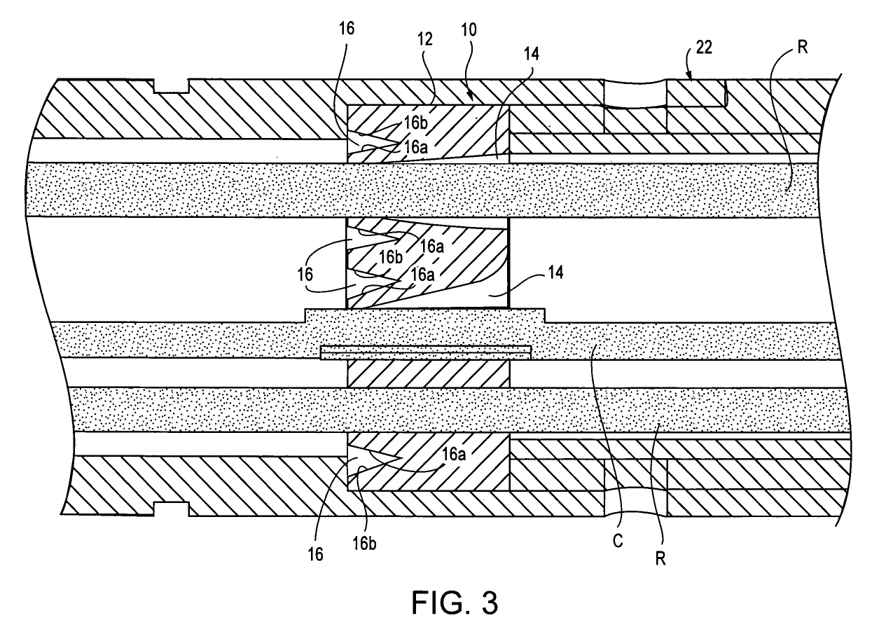

Referring to FIG. 2, the seal 10 is shown placed in the instrument or device 22 from a cross-sectional view. FIG. 2 is shown with four receiving apertures 14 through which apparatus C, R move in the axial direction X. The channels 16 may be seen from this view around each receiving aperture or set of apertures 14. These features can also be seen in FIG. 3 which is an enlarged view of FIG. 2.

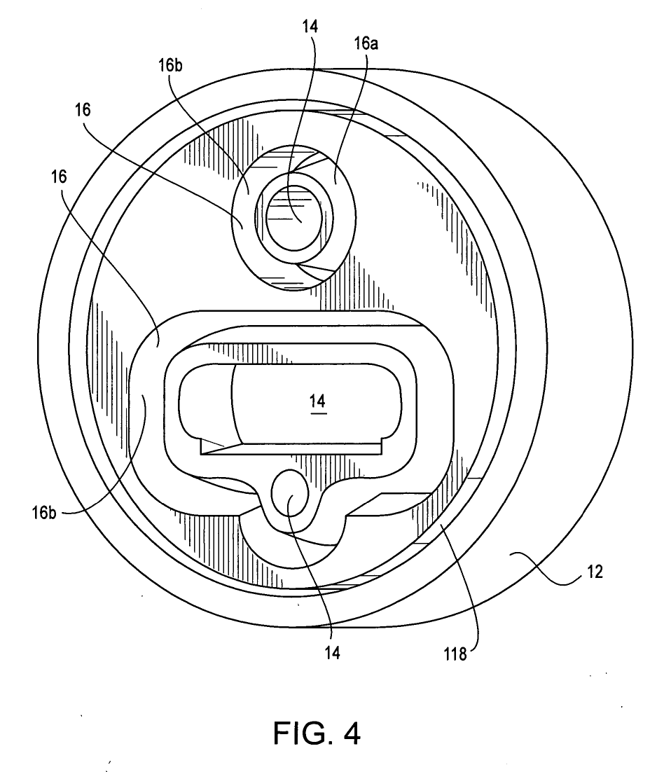

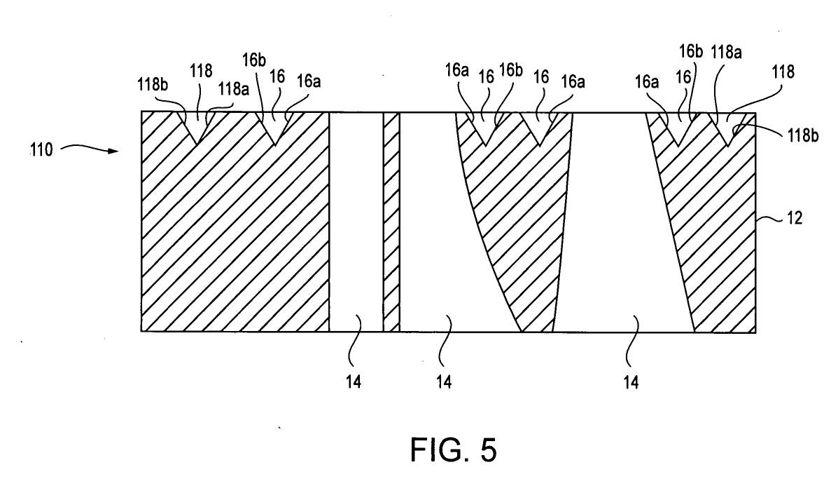

Another, second embodiment of the present invention is shown in FIGS. 4-5. The seal 110 includes an edge surface 12, receiving apertures 14, channels 16, and an edge channel 118. Similar to the previously-described channels 16 around the receiving apertures 14, the edge channel 118 further has an inner wall portion 11 8a and an outer wall portion 11 8b and is constructed so that the outer wall portion 11 8b presses upon the inner surface of the instrument 22 when under pressure from fluid. Also similar to the channels 16, the edge channel 118 is shown as generally V shaped but can be any shape. FIG. 5 shows a cross-sectional view of the embodiment in FIG. 4.

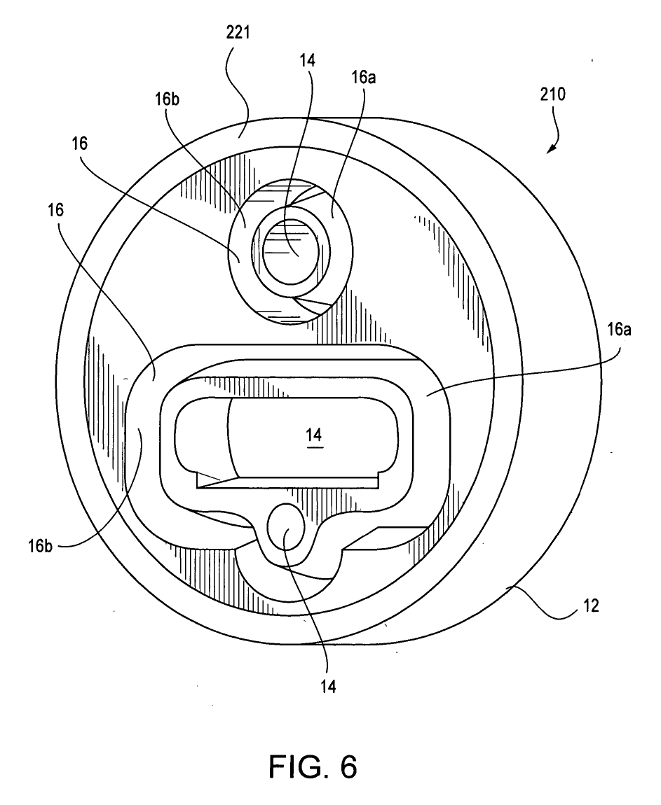

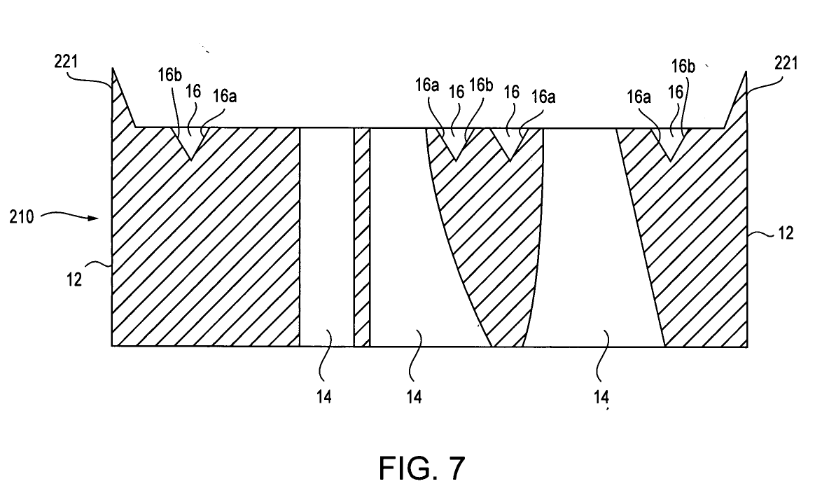

A third, further embodiment of the present invention is shown in FIGS. 6-7. The seal 210 of this embodiment is similar to that shown in FIG. 1 except that the an edge lip 221 has been added. The edge lip 221 increases the contact area between the edge surface 12 and the inner surface of the device 22, and outwardly presses against the inner surface of the device when under pressure from fluid or air. The edge lip 221 is shown as being an angular or tapered shape but it can be any shape that allows for the necessary configuration. FIG. 7 shows a cross-sectional view of the embodiment in FIG. 6.

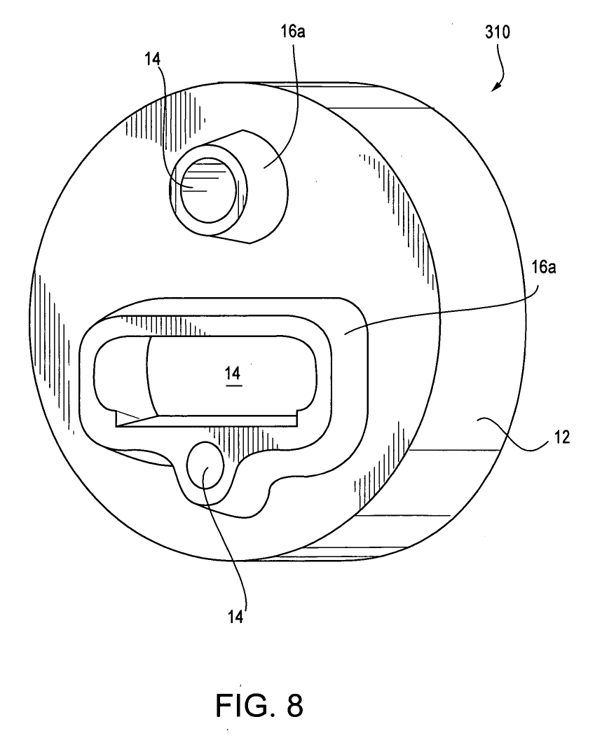

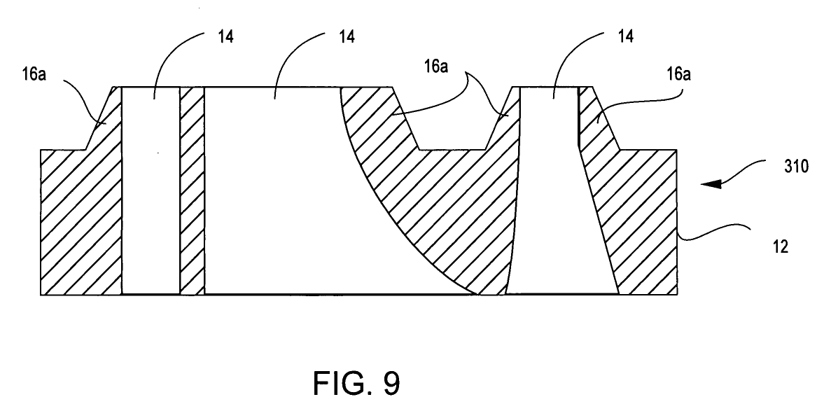

FIGS. 8-9 show another embodiment of a seal 310 according to the present invention. The seal is similar to the above three embodiments, except the there are no channels surrounding the receiving apertures 14. Rather, the inner lip portion 16a is present and the outer lip portion 16b is absent. It is noted that this embodiment may be combined with features of the above three embodiments. For example, the edge channel 118 of the second embodiment may be provided and/or the edge lip 221 of the third embodiment may be provided.

It is noted that the foregoing examples have been provided merely for the purpose of explanation and are in no way to be construed as limiting of the present invention. While the present invention has been described with reference to a preferred embodiment, it should be understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. An elastomeric internal seal for preventing or minimizing fluid from passing from a first end of a tubular instrument to a second end of the tubular instrument, the seal comprising:
an edge portion configured to contact an inner surface of the instrument;
at least one receiving aperture configured to receive an apparatus therethrough, the apparatus extending in a generally longitudinal axial direction in relation to the tubular instrument; and
a generally V-shaped channel generally extending about a circumference of said at least one receiving aperture, said generally V-shaped channel configured such that pressure created by the fluid causes one wall of said generally V-shaped channel to press against the apparatus.

2. The seal according to claim 1, wherein both walls of each said V-shaped channel are obliquely angled in relation to the generally longitudinal direction.

3. The seal according to claim 1, wherein said receiving aperture is configured to allow the apparatus to move in the axial direction.

4. The seal according to claim 1, further comprising an ethylene propylene diene monomer.

5. The seal according to claim 1, further comprising a generally V-shaped edge channel generally extending about said edge portion, said edge channel configured such that pressure created by the fluid causes one wall of said edge channel to press against the inner surface of the instrument.

6. The seal according to claim 1, further comprising a tapered lip generally extending about said edge portion, said tapered lip configured such that pressure created by the fluid causes said tapered lip to press against the inner surface of the instrument.

7. An elastomeric internal seal for preventing or reducing fluid from passing from a first end of a tubular instrument to a second end of the tubular instrument, the seal comprising:
an edge portion configured to contact an inner surface of the instrument;
at least one receiving aperture configured to receive an apparatus therethrough, the apparatus extending in a generally longitudinal direction in relation to the tubular instrument; and
a first lip portion channel generally extending toward the first end and about a circumference of said at least one receiving aperture, wherein said first lip portion is configured such that pressure created by the fluid causes said first lip portion to press against the apparatus.

8. The seal according to claim 7, wherein said first lip portion is tapered.

9. The seal according to claim 7, wherein said first lip portion is obliquely angled in relation to the generally longitudinal direction

10. The seal according to claim 7, further comprising a second lip portion generally extending toward the first end and about a circumference of and facing said first lip portion.

11. The seal according to claim 10, wherein at least one of said first lip portion or said second lip portion is tapered.

12. The seal according to claim 10, wherein at least one of said first lip portion or said second lip portion is obliquely angled in relation to the generally longitudinal direction.

13. The seal according to claim 10, wherein said first lip portion and said second lip portion form a generally V-shaped channel.

14. The seal according to claim 7, wherein said receiving aperture is configured to allow the apparatus to move in the axial direction.

15. The seal according to claim 7, further comprising an ethylene propylene diene monomer.

16. The seal according to claim 10, further comprising an edge lip generally extending about said edge portion, wherein said edge lip is configured such that pressure created by the fluid causes said edge lip to press against the inner surface of the instrument.

17. The seal according to claim 16, wherein said edge lip is tapered.

18. A medical instrument comprising:
a tubular portion having a first end and a second end, said first end configured to be inserted into a body cavity;
an apparatus generally disposed within said tubular portion and extending in a generally longitudinal direction in relation to the tubular, said apparatus configured to effect an operation relating to the body cavity
an elastomeric internal seal disposed in said tubular portion and configured to prevent fluid from passing from said first end to said second end, said seal comprising:
an edge portion configured to contact an inner surface of said tubular portion;
at least one receiving aperture configured to receive the apparatus therethrough; and
a first lip portion channel generally extending toward the first end and about a circumference of said at least one receiving aperture, wherein said first tapered lip portion is configured such that pressure created by the fluid causes said first tapered lip portion to press against the apparatus.

19. The instrument according to claim 18, wherein said seal further comprises a second lip portion generally extending toward the first end and about a circumference of and facing said first lip portion.

20. The instrument according to claim 19, wherein at least one of said first lip portion or said second lip portion is tapered.
